(19) United States
(12) Patent Application Publication
(10) Pub. No.: US 2022/0118200 A1
(43) Pub. Date: Apr. 21, 2022

(54) Discrete Inhaling Device

(71) Applicant: Drury Industries, LLC, Long Grove, IL (US)

(72) Inventors: Clay L. Drury, Long Grove, IL (US); Kelly Drury, Long Grove, IL (US)

(73) Assignee: Drury Industries, LLC, Long Grove, IL (US)

(21) Appl. No.: 17/074,177

(22) Filed: Oct. 19, 2020

(51) Int. Cl. A61M 15/06; A61M 11/04; A61M 15/00

(52) U.S. Cl. CPC A61M 15/06; A61M 11/041; A61M 2205/8206; A61M 15/0028; A61M 15/0025

(57) ABSTRACT

A discrete inhalation device comprising a cartridge and a modular body disguised as a lip applicator such as lip gloss or lip stick.

### FIELD OF THE INVENTION

This invention generally relates to consumer products designed for the use of nicotine, cannabis or related products, and more particularly to devices and methodologies for the discrete use of such products.

### BACKGROUND OF THE INVENTION

Inhaling devices such as vaporizers, vaporizing pens, and vaporizing machines are used to vaporize substances such as tobaccos, oils, liquids, medical drugs, and plant-based oils and herbs. Once vaporized, these substances are then inhaled by consumers. Using vaporizers or vape pens has become a popular method for inhaling tobacco-based and herbal products such as, for example, cannabis. The most common method of using a vaporizer involves attaching a cartridge to a battery unit. These cartridges contain the oil (or liquid, wax or solid plant material) reservoir, a vaporizer or atomizer unit, a mouthpiece and a battery connector. The oil reservoir contains the substance that is heated and vaporized, while the vaporizer gets electric power from the battery using the battery connector and heats the substance to a specific temperature. The battery connector is typically afforded through a thread to the battery unit, however, other types of connections exist as well, such as magnetic or snap-fit connections.

Vaporizers traditionally come in shapes approximating a cigarette or cigar. As such, most vaporizers have a configuration that resembles the long, tubular shape of a cigarette. The shape of the smoking simulator allows the user to have an overall experience similar to smoking a cigarette while not delivering the carcinogenic substances created by burning tobacco or plant oils and inhaling the smoke. Although the shape of these devices is specifically selected to replicate a cigarette, users often suffer from the same anti-smoking stigmatism associated with actually smoking tobacco cigarettes.

Recently, vaporizers with various shapes are disclosed that disguise the device as something other than an inhalation device. For example, U.S. Pat. No. 9,233,217 discloses a water bottle or a drinking cup with a straw, while U.S. Pat. No. 10,278,426 teaches a personal vapor device shaped as a credit card such that it fits into a wallet. However, these devices either are too large or reveal their true nature as a personal vaping device when said device approaches the users mouth for inhalation. Other technology, such as that disclosed in U.S. Pat. No. 10,595,562, has focused on inhalation devices for inhaling a vaporized substance that employs sensors that sense the vaporized substance and a processor that utilizes data from the sensor to meter the amount consumed. While these devices afford the user with a precise dose-control mechanism, they do not provide a casual user with the privacy necessary when out in public.

There therefore still exits a need for creating a discrete inhalation device that does not call attention to the user. The device should easily fit into the pocket of an article of clothing when not in use, while maintaining its disguise as the user approaches the device to her or his lips for use. Accordingly, an inhalation device is desired that mimics the shape and appearance of a lip applicator, such as for example, lip gloss or lip stick. The device can have a modular body that looks like the body of a lip applicator, including incorporation of a substance dose-control mechanism that does not reveal the identity of the device.

### SUMMARY OF THE INVENTION

In light of the foregoing, it is an object of the present invention to provide an inhalation device that is designed to look like a lip applicator that is compact enough to fit into, for example, a pocket to an article of clothing, while maintaining its disguise as a lip applicator when a user inhales a vaporized substance from the device. It will be understood by those skilled in the art that one or more aspects of this invention can meet certain objectives, while one or more other aspects can meet certain other objectives. Each objective may not apply equally, in all its respects, to every aspect of this invention. As such, the following objects can be viewed in the alternative with respect to any one aspect of this invention.

Accordingly, it can also be an object of the present invention to provide a modular body for an inhalation device for inhaling a vaporized substance, the modular body comprising a top end and a bottom end and an outside portion and an inside portion, wherein the outside portion is clear and the inside portion is colored such that the inside portion is at least partially visible through the outside portion; a first connecting mechanism on the top end of the modular body or on a power source housed inside the modular body for engaging a cartridge, the cartridge comprising a mouthpiece, an interior filled with a substance in an unvaporized form, a heating element for vaporizing the substance to a vaporized form, and a second coupling means on the cartridge for engaging the first connecting mechanism on the top of the modular body or on the power source; and a cap removably-couplable to the top end of the modular body, the cap covering the cartridge when coupled to the top end of the modular body.

In part, it is further an object of the invention to provide a modular body for an inhalation device for inhaling a vaporized substance, the modular body comprising a top end and a bottom end and an outside portion and an inside portion, wherein the outside portion is clear and the inside portion is colored such that the inside portion is at least partially visible through the outside portion; a first connecting mechanism on the top end of the modular body or on a power source housed inside the modular body for engaging a cartridge, the cartridge comprising a mouthpiece, an interior filled with a substance in an unvaporized form, a heating element for vaporizing the substance to a vaporized form; a second coupling means on the cartridge for engaging the first connecting mechanism on the top of the modular body or on the power source housed inside the modular body; and a cap removably-couplable to the top end of the modular body, the cap covering the cartridge when coupled to the top end of the modular body; a dose-control mechanism for the vaporized form of the substance; and a cap that fits over the cartridge when the cartridge is engaged with the body, the cap being removably-couplable to the top end of the body.

As provided herein, the dose control mechanism can comprise a sliding member disposed slidably in a recess on an outer surface of the modular body and configured to move axially about the modular body between a first position and a second position when the cap is not coupled to the top end of the modular body, the sliding member having a width substantially equivalent to a width of the recess, wherein the first position allows the vaporized form of the substance to flow freely in a direction from the interior of the cartridge to the mouthpiece of the cartridge as the user is inhaling through the mouthpiece, wherein the second position is a time-controlled shut-off of flow of the vaporized form of the substance in the direction from the interior of the cartridge to the mouthpiece of the cartridge, and wherein only one of the first position and the second position is afforded when the cap is coupled to the top end of the modular body, the sliding member having a length substantially equivalent to a length of the recess when the cap is coupled to the top end of the modular body; and a time-controlled shut-off component engaged to the sliding member and housed inside the modular body.

Other objects, features, benefits and advantages of the present invention will be apparent from this summary and the following descriptions of certain embodiments and will be readily apparent to those skilled in the art having knowledge of discrete inhalation devices. Such objects, features, benefits and advantages will be apparent from the above as taken into conjunction with the accompanying examples, figures and all reasonable inferences to be drawn therefrom. The disclosures in this application of all articles and references, including patents, are incorporated herein by reference.

### BRIEF DESCRIPTION OF THE DRAWINGS

FIG. 1 is a side view of a modular body for an inhalation device as disclosed herein.

FIG. 2 is another side view of the modular body for an inhalation device in FIG. 1 with the cap removed.

FIG. 3 is the same side view as FIG. 2 of the modular body for an inhalation device with a cartridge initially engaged.

FIG. 4 is the same side view as FIG. 2 of the modular body for an inhalation device with a cartridge fully engaged.

FIG. 5 is a side view of the inhalation device of FIG. 4 with the cap covering the mouthpiece of the cartridge.

FIG. 6 is a perspective view of a user manipulating the sliding member portion of a dose-control mechanism with the inhalation device of FIG. 4.

FIG. 7 illustrates the axial movement between a first and second position of a sliding member for a dose control mechanism for the inhalation device of FIG. 4.

FIG. 8 depicts a user with the discrete inhalation device as described herein.

### DETAILED DESCRIPTION OF INVENTION

As it relates to certain non-limiting embodiments, the present invention relates to a modular body for an inhalation device for inhaling a vaporized substance comprising: a top end and a bottom end and an outside portion and an inside portion, wherein the outside portion is clear and the inside portion is colored such that the inside portion is at least partially visible through the outside portion; a first connecting mechanism either on the top end of the modular body or on a power source housed inside the modular body, the first connecting mechanism configured to engage a cartridge, the cartridge comprising a mouthpiece, an interior filled with a substance in an unvaporized form, a heating element for vaporizing the substance to a vaporized form, and a second coupling means on the cartridge for engaging the first connecting mechanism; and a cap removably-couplable to the top end of the modular body, the cap covering the cartridge when coupled to the top end of the modular body.

As used herein, “inhalation device” is a device comprising a cartridge and a modular body. As discussed in detail below in the specific non-limiting embodiments of the invention, the cartridge of the inhalation device preferably comprises a reservoir or interior for an oil, liquid, wax or solid plant material, a heating element, such as for example, a vaporizer or atomizer unit, a mouthpiece and optionally a power source connector. The reservoir contains the substance that is heated, vaporized an ultimately inhaled by a user. The heated substance is preferably nicotine or cannabis, more preferably cannabis, but can be a variety of other small molecules that can be vaporized, or combinations thereof, such as for example, flavonoids, terpenes and the like. Preferably, the vaporizer gets electric power from a battery power source using a battery connector. The vaporizer, in turn, heats the substance to a specific temperature. The power source, which is preferably housed in the modular body as described herein, and the cartridge are configured to be mechanically and/or electrically coupled to one another, and more preferably both mechanically and electrically coupled to one another. For example, the cartridge can comprise an electrically conductive threaded fastener, such as for example, a 510 connector, and the power source can comprise an electrically conductive threaded receiver. By “510 connector” is meant a mechanical and electrical connector that runs 5 mm long and has 5 threads on the male connection, which is preferably on the cartridge end, and 5 threads on the female connection, which is preferably on the battery, which, as disclosed herein, is housed in the modular body. The threaded fastener and the threaded receiver can be coupled to one another, thereby mechanically and electrically coupling the power source with the cartridge. Under such a mechanism, the threaded receiver is part of or connected to the power source, preferably a battery, and the threaded fastener is part of or connected to the heating element. It is to be understood by those skilled in the art that other types of connection mechanisms for the power source and cartridge are contemplated, such as for example, magnetic connection mechanisms, snaps, twist caps, push on connections, gasketed connections, quick connection mechanisms, turn lock mechanisms, t-bar connections, and the like.

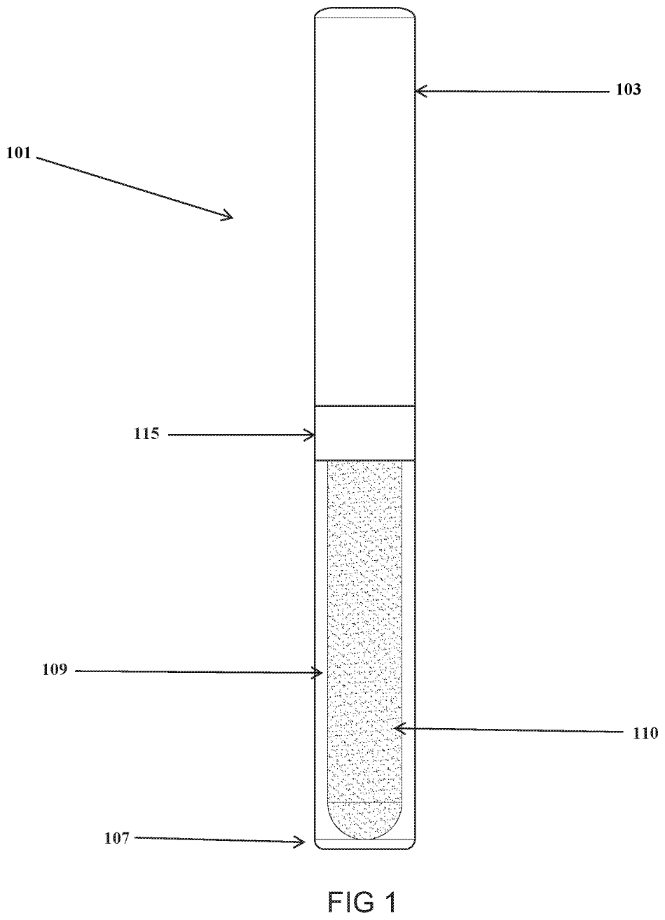

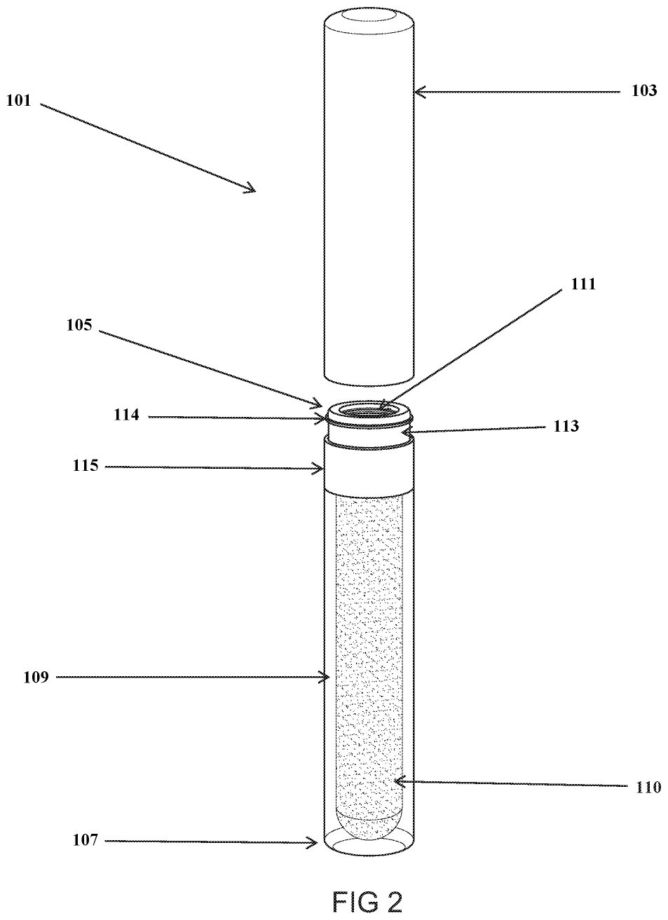

Referring now to the specific non-limiting embodiments of the invention, FIGS. 1-5 depict a side view of a modular body 101 for an inhalation device. The modular body 101 is disguised as a lip applicator, preferably as lip gloss or lip stick, and more preferably as lip gloss. As such, the modular body 101 takes on the shape of a lip gloss or a lip stick container, preferably either a tubular or a rectangular shape, and comprises a cap 103 which can either be coupled to a top end 105 of the modular body 101, as shown in FIG. 1, or removed from the top end 105 of the modular body 101, as depicted in FIG. 2. The cap 103 can be removably-coupled to the top end 105 of the modular body 101 in a variety of ways, preferably by a snap-fit design and more preferably by an annular snap-fit (or snap-on) design. The modular body 101 also has a bottom end 107 which is preferably sealed, and more preferably permanently sealed. The modular body 101 additionally features a clear, outside portion 109 and a colored, inside portion 110, such that the inside portion 110 is at least partially visible through the outside portion 109. Again, the outside portion/inside portion 109/110 is designed to resemble a lip applicator container, such as for example, a lip gloss container or a lip stick container (the outer portion 109), and its contents (the inner portion 110). The outside portion 109 and the inside portion 110 can be one layer, or a separate layer for each of the outside portion 109 and the inside portion 110. Preferably, the outside portion 109 is made of glass or an at least partially clear plastic, while the inside portion 110 is made of the same or different material as the outer portion 109 while also preferably made of glass or plastic. More preferably, the outer portion 109 is made of a clear transparent plastic or a plastic with a matte, frosty finish. Either way, the outer portion 109 is configured to make the colored inner portion 110 at least partially visible through the inner portion 109.

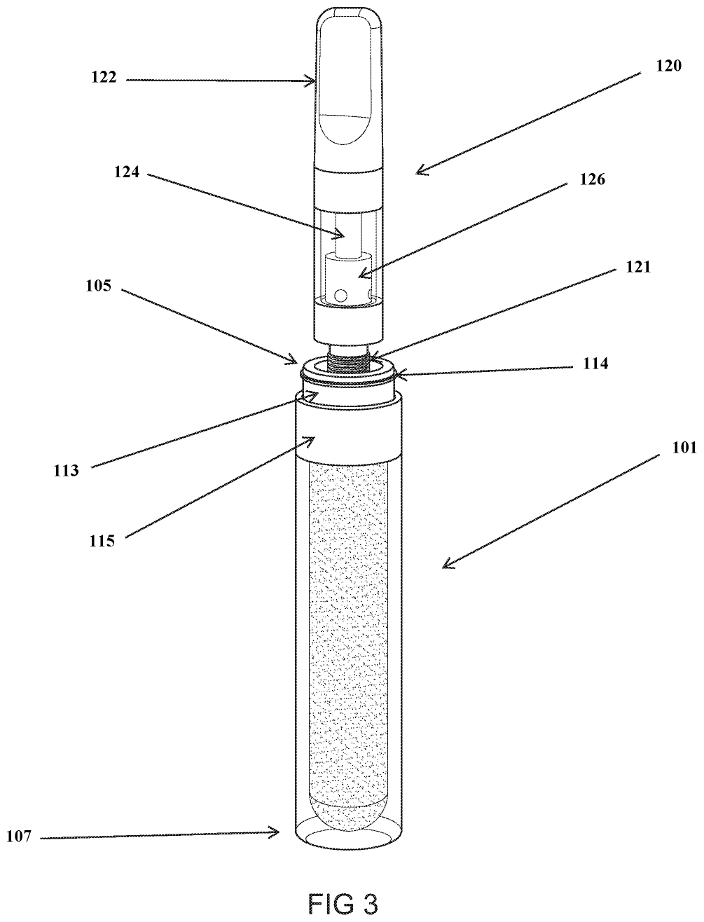

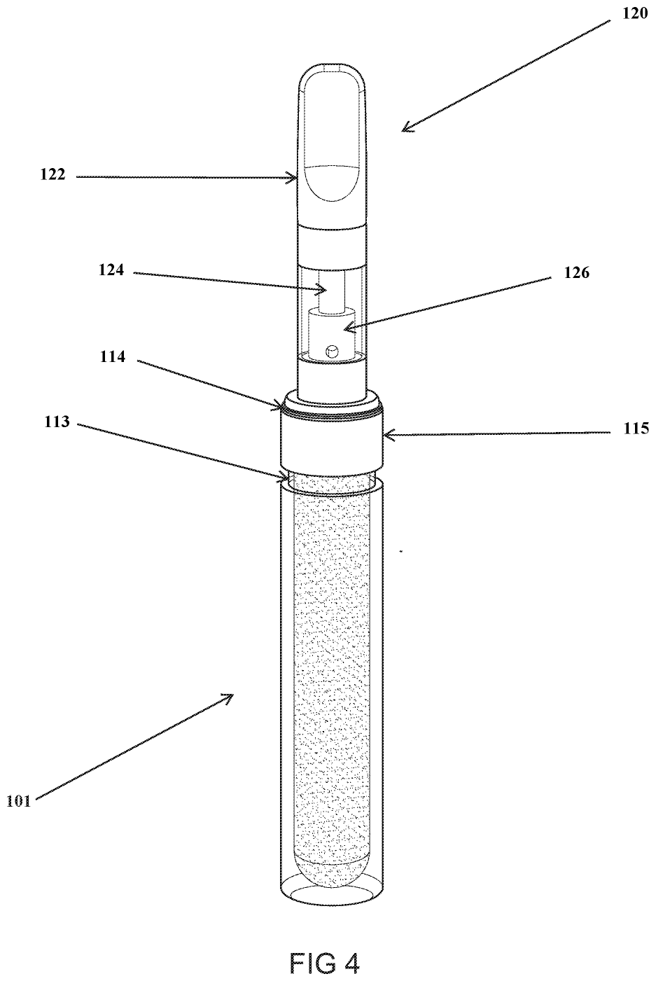

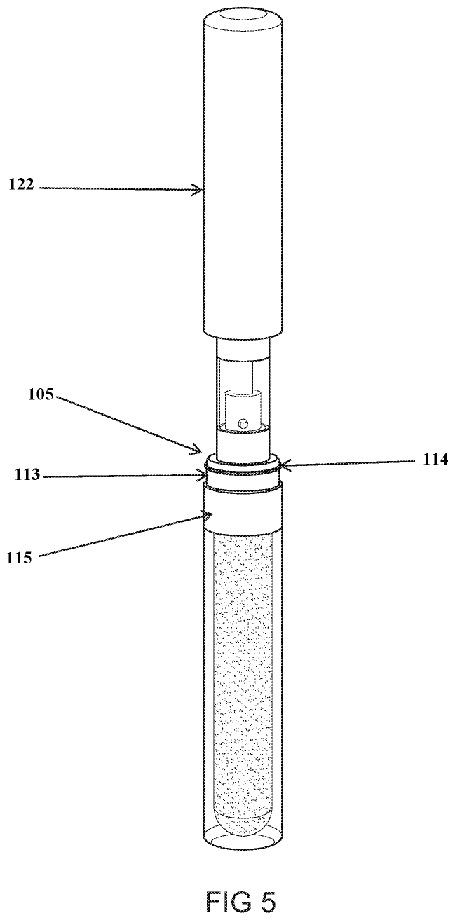

As noted above, the power source (not pictured), which is preferably housed in the modular body, and the cartridge are configured to be mechanically and/or electrically coupled to one another, and more preferably both mechanically and electrically coupled to one another, wherein the connecting mechanism is preferably by threads. FIG. 2 depicts, as an example, a first set of threads 111 on the inside of the top end 105 of the modular body 101 for engaging a cartridge 120. FIG. 3 shows the cartridge 120 partially engaged to the modular body 101 and comprising a mouthpiece 122, a reservoir or interior 124 that can be filled with a substance such as but not limited to nicotine or cannabis (or certain small molecules extracted or isolated from cannabis) in an unvaporized form, a heating element 126 for vaporizing the substance to a vaporized form, and a second set of threads 121 on the outside of the cartridge 120 for engaging the first set of threads 111 on the modular body 101. Preferably, the second set of threads on the outside of the cartridge are on the heating element directly. FIG. 4 depicts the cartridge 120 fully engaged with the modular body 101. The cap 103 covers the cartridge 120 when coupled to the top end 105 of the modular body 101, as shown in FIG. 5.

As shown in FIG. 3 and FIG. 4, the outside portion 109 can extend longitudinally throughout the modular body 101 from the bottom end 107 to a recess 113 near the top end 105. Similarly, the inside portion 110 can extend longitudinally throughout the modular body 101 from or near the bottom end 107 to a recess 113 near the top end 105. Preferably, the inner portion 110 can extend longitudinally from the bottom end 107 and past the outer portion 109 to an annular ridge 114 on or near the top end 105 of the modular body 101. The recess 113 can therefore be defined as that part of the inner portion 110, longitudinally, from the annular ridge 114 to where the outer portion 109 ends proximate to the top end 105.

Disposed within the recess 113 can be a sliding member 115 in the shape of, for example, a ring, similar to that found on the top of a tubular lip gloss or lip stick container and below a lip gloss or lip stick cap when the cap is coupled to said container. It is to be understood that the sliding member 115 can also take the form of other shapes, such as rectangular, for use on rectangular lip applicator devices. The sliding member 115, as disclosed herein, can be part of a dose-control mechanism (collectively not pictured) for a vaporized form of the substance to be inhaled. The dose-control mechanism can comprise the sliding member 115, as a switch that can be operated by a user, and a time-controlled shut-off component (not pictured) engaged to the sliding member 115 and housed inside the modular body 101. As depicted in FIG. 3 and FIG. 4, the sliding member 115 can have a width that is substantially equivalent to that of the recess 114. Further, as shown in FIG. 1, the sliding member 115 can have a longitudinal length substantially equivalent to a length of the recess 113 when the cap 103 is coupled to the top end 105 of the modular body 101.

The sliding member 115 is configured to move axially along the modular body 101 in the recess 114 when the cap 103 is removed. Specifically, the sliding member 115 can move axially into a position, as shown in FIG. 3 and FIG. 5, which either allows the vaporized form of the substance to flow freely in a direction from the reservoir 124 of the cartridge 120 to the mouthpiece 122 of the cartridge 120 as the user is inhaling through the mouthpiece 122, or provides for a time-controlled shut-off of flow of the vaporized form of the substance in the direction from the reservoir 124 to the mouthpiece 122 as the user is inhaling. FIG. 4 shows the sliding member in a second position, which allows for the other of either the vaporized form of the substance to flow freely in a direction from the reservoir 124 of the cartridge 120 to the mouthpiece 122 of the cartridge 120 as the user is inhaling through the mouthpiece 122, or provides for a time-controlled shut-off of flow of the vaporized form of the substance in the direction from the reservoir 124 to the mouthpiece 122 as the user is inhaling.

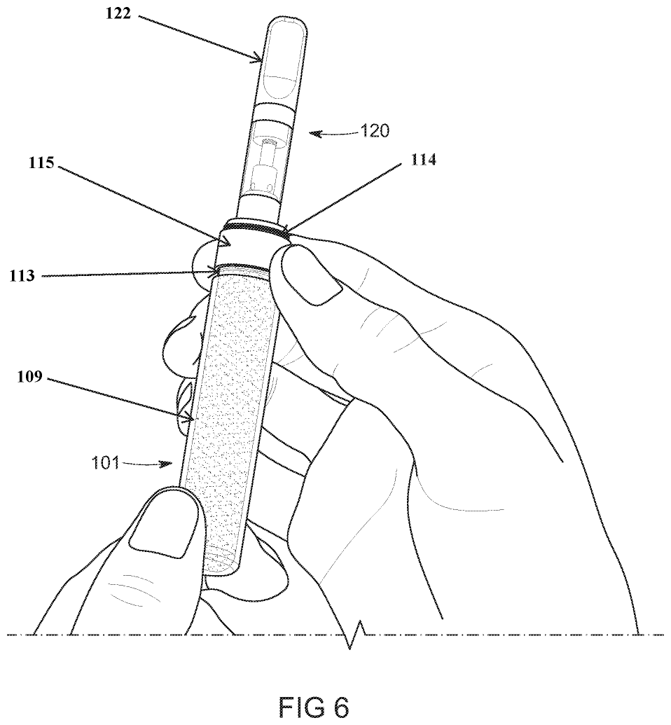

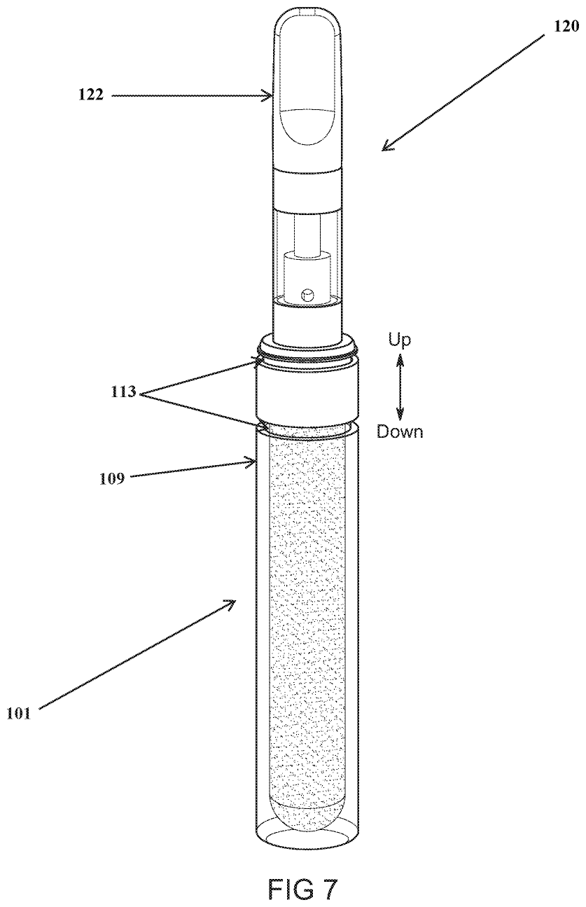

In other words, as depicted in FIG. 6, a user can either slide the sliding member 115 with her or his fingers axially within the recess 113 in a direction towards the mouthpiece 122 and against the annular ridge 114 or in a direction away from the mouthpiece 122 of the cartridge 120 and against where the outer portion 109 of the modular body 101 ends. As discussed above, the two different positions provide for either a steady-stream of inhalation of vaporized substance or a time-controlled shut-off of the vaporized substance when the user is inhaling through the mouthpiece 122. FIG. 7 depicts the in-between axial movement of the sliding member 115 within the recess 113 as either “up” towards the mouthpiece 122 of the cartridge 120 or “down” towards the part of the recess 113 where the outer portion 109 of the modular body 101 ends.

The time-controlled shut-off component as described herein can be various mechanisms or combinations thereof as known in the art. For example, the component can include a valve, in which case the reservoir may be a propellant pressurized reservoir, and a valve-opening/closing actuator is provided to operate the valve for delivering the metered substance (as a liquid or an oil) from the propellant pressurized reservoir to the heating element. Alternatively, the time-controlled shut-off component can include a pump with a pump actuating mechanism to operate the pump for delivering the metered substance (as a liquid or an oil) from the propellant pressurized liquid reservoir to the heating element. In addition, a processor along with voltage measurements, current measurements, a vapor analyzer and a microphone (for listening to the white noise passing through) can be employed. As used herein, the sliding member can be used with one or more of the above-mentioned devices for actuating the heating element's vaporization of delivered, metered substance (as a liquid or an oil). In this way, the sliding member can act as a user-activated switch for closing an electrical circuit connecting a battery and the heating element.

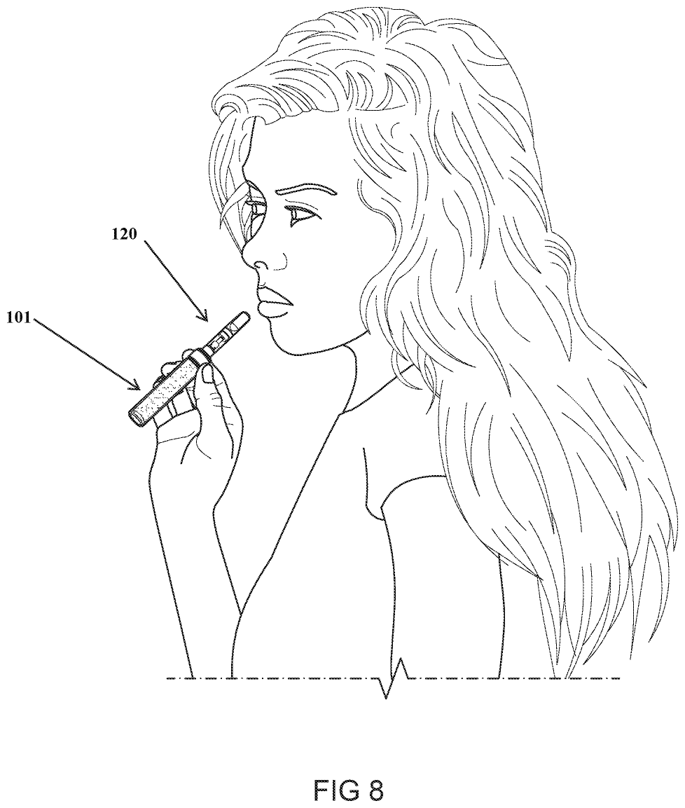

It is therefore an advantage of the inhalation device described herein (see FIG. 8) to provide a discrete inhalation device. The inhalation device comprises a cartridge as described above and a modular body disguised as a lip applicator container, such as a lip gloss container or a lip stick container, with a cap. The inhalation device described herein therefore remains discrete when the cap is on or whether the cap is off and a user approaches her or his lips (or even reaches the lips to inhale) with the device, giving the appearance of someone applying lip gloss or lip stick to her lips, as depicted in FIG. 8. Further, the modular body of the discrete inhalation device can also incorporate a time-dependent dose control mechanism with a switch disposed on the modular body that also helps give the inhaling device the appearance of a lip applicator. The inhalation device described herein is also small enough in size to fit into a purse or pocket of an article of clothing.

Although various embodiments of the inhalation device are illustrated and described herein, it is appreciated that other embodiments are contemplated as being within the scope of the present disclosure and that various substitutions may be made by those skilled in the art without departing from the spirit of the same. As described herein, the discrete inhalation device conceals said device by having a modular body that has the appearance of another article, such that others in proximity of a user of the discrete inhalation device can not identify the use of the same.

What is claimed is:

1. A modular body for an inhalation device for inhaling a vaporized substance, comprising:
a top end and a bottom end and an outside portion and an inside portion, wherein the outside portion is clear and the inside portion is colored such that the inside portion is at least partially visible through the outside portion;
a first connecting mechanism either on the top end of the modular body or on a power source housed inside the modular body, the first connecting mechanism configured to engage a cartridge, the cartridge comprising a mouthpiece, an interior filled with a substance in an unvaporized form, a heating element for vaporizing the substance to a vaporized form, and a second coupling means on the cartridge for engaging the first connecting mechanism; and
a cap removably-couplable to the top end of the modular body, the cap covering the cartridge when coupled to the top end of the modular body.

2. The modular body of claim 2, wherein the first connecting mechanism is a first set of threads on an inside surface of the top end of the modular body or on the power source, and the second connecting mechanism is a second set of threads on an outside surface of the cartridge for engaging the first set of threads.

3. The modular body of claim 1, wherein the modular body has a dose-control mechanism for the vaporized form of the substance, the dose control mechanism comprising:
a sliding member disposed slidably in a recess on an outer surface of the modular body and configured to move axially about the modular body between a first position and a second position when the cap is not coupled to the top end of the modular body, the sliding member having a width substantially equivalent to a width of the recess, wherein the first position allows the vaporized form of the substance to flow freely in a direction from the interior of the cartridge to the mouthpiece of the cartridge as the user is inhaling through the mouthpiece, wherein the second position is a time-controlled shut-off of flow of the vaporized form of the substance in the direction from the interior of the cartridge to the mouthpiece of the cartridge, and wherein only one of the first position and the second position is afforded when the cap is coupled to the top end of the modular body, the sliding member having a length substantially equivalent to a length of the recess when the cap is coupled to the top end of the modular body; and
a time-controlled shut-off component engaged to the sliding member and housed inside the modular body.

4. The modular body of claim 1, wherein the inner portion and outer portion of the modular body takes on a tubular shape or a rectangular shape.

5. The modular body of claim 4, wherein the inner portion and the outer portion of the molecular body takes on a tubular shape.

6. The modular body of claim 1, wherein the heating element of the cartridge that engages the modular body is a vaporizing or atomizing unit.

7. The modular body of claim 2, wherein the power source is a battery housed in the modular body.

8. The modular body of claim 7, wherein the first set of threads is on the battery.

9. The modular body of claim 8, wherein the second set of threads on the outside surface of the cartridge is on the heating element.

10. The modular body of claim 9, wherein the first set of threads and the second set of threads form both a mechanical and an electrical couple when the first set of threads are engaged with the second set of threads.

11. A modular body for an inhalation device for inhaling a vaporized substance, comprising:
a top end and a bottom end and an outside portion and an inside portion, wherein the outside portion is clear and the inside portion is colored such that the inside portion is at least partially visible through the outside portion;
a first connecting mechanism either on the top end of the modular body or on a power source housed inside the modular body, the first connecting mechanism configured to engage a cartridge, the cartridge comprising a mouthpiece, an interior filled with a substance in an unvaporized form, a heating element for vaporizing the substance to a vaporized form, and a second coupling means on the cartridge for engaging the first connecting mechanism;
a cap removably-couplable to the top end of the modular body, the cap covering the cartridge when coupled to the top end of the modular body; and
a dose-control mechanism for the vaporized form of the substance, the dose control mechanism further comprising:
a sliding member disposed slidably in a recess on an outer surface of the modular body and configured to move axially about the modular body between a first position and a second position when the cap is not coupled to the top end of the modular body, the sliding member having a width substantially equivalent to a width of the recess, wherein the first position allows the vaporized form of the substance to flow freely in a direction from the interior of the cartridge to the mouthpiece of the cartridge as the user is inhaling through the mouthpiece, wherein the second position is a time-controlled shut-off of flow of the vaporized form of the substance in the direction from the interior of the cartridge to the mouthpiece of the cartridge, and wherein only one of the first position and the second position is afforded when the cap is coupled to the top end of the modular body, the sliding member having a length substantially equivalent to a length of the recess when the cap is coupled to the top end of the modular body; and
a time-controlled shut-off component engaged to the sliding member and housed inside the modular body.

12. The modular body of claim 11, wherein the inner portion and outer portion of the modular body takes on a tubular shape or a rectangular shape.

13. The modular body of claim 12, wherein the inner portion and the outer portion of the molecular body takes on a tubular shape.

14. The modular body of claim 11, wherein the heating element of the cartridge that engages the modular body is a vaporizing or atomizing unit.

15. The modular body of claim 14, wherein the power source is a battery housed in the modular body.

16. The modular body of claim 15, wherein the first set of threads is on the battery.

17. The modular body of claim 16, wherein the second set of threads on the outside surface of the cartridge is on the heating element.

18. The modular body of claim 17, wherein the first set of threads and the second set of threads form both a mechanical and an electrical couple when the first set of threads are engaged with the second set of threads.
